(19) United States
(12) Patent Application Publication
(10) Pub. No.: US 2026/0283193 A1
(43) Pub. Date: Sep. 24, 2026

(54) GLUCOAMYLASE AS BINDING AGENT FOR NON-ANIMAL PROTEIN FOOD PRODUCTS

(71) Applicant: DSM IP Assets B.V., Maastricht (NL)

(72) Inventor: Petrus Jacobus Theodorus DEKKER, ECHT (NL)

(21) Appl. No.: 19/474,226

(22) PCT Filed: Apr. 11, 2024
(86) PCT No.: PCT/EP2024/059768

§ 371 Date: Oct. 10, 2025

(30) Foreign Application Priority Data
Apr. 12, 2023 (EP) 23167525.7

(51) Int. Cl. A23J 3/34; A23J 3/22; A23L 29/269; C12N 9/34

(52) U.S. Cl. CPC A23J 3/347; A23J 3/227; A23L 29/269; A23L 29/273; C12N 9/2428; C12Y 302/01003

(57) ABSTRACT

The present invention relates to the field of food. Herein disclosed is a method for preparing a non-animal protein food product or a cultured meat food product comprising adding a thermolabile glucoamylase protein preparation to an ingredient used in the preparation of said food product and processing the obtained glucoamylase protein preparation and ingredient composition into a non-animal protein food product or a cultured meat food product.

### FIELD

The present invention relates to the field of food.

### BACKGROUND

Food products comprising non-animal proteins (for example plant proteins) as alternative to animal-derived proteins nowadays receive attention because of consumer concerns about the environmental impact of animal-based products and the beneficial nutritional characteristics of non-animal protein based foods. In particular, beverages based on plant proteins as alternative to dairy products such as milk, yoghurt or ice cream have gained popularity.

The many disadvantages associated with the use of animal-derived protein for human consumption, ranging from acceptability of raising animals for consumption to the fact that such meat production is inefficient in terms of feed input to food output and carbon footprint, makes the ongoing search for improved meat alternatives one of the most active food developments in present day society.

Historically, meat alternatives achieve a certain protein content using fermented vegetable sources such as soy (e.g. tofu, tempeh) or gluten/wheat (e.g. seitan). Today, modern techniques as extrusion and 3D-printing are used to make meat alternatives with more meat-like texture, flavor and appearance. Soy and gluten are still favorable sources for such meat alternatives because they are widely available, affordable, relatively high in protein and well processable. However, GM-produced soy and gluten intolerance or allergenicities are triggering consumer demands for alternatives. Producers of meat alternatives turn to other proteins, for example like those derived from legumes, e.g., pea. However, use of these alternative protein sources is accompanied with new problems. The protein mixtures are often not as easily processible as the traditional soy or gluten or their combinations, and in many cases also lead to texturized food proteins that do not mimic the nutrition, texture, appearance, and/or the taste of animal-derived meat products. As a result, consumers typically consider such meat alternatives unappealing and unpalatable. Hence, there is a need in the art for meat alternatives that are appealing and palatable.

A commonly used binder, or binding agent, in dairy- or meat alternatives is methyl cellulose. Methyl cellulose is undesired in view of the food label requirements requiring mentioning of such chemical. Consumers more and more desire clean label food products. Other common binders are wheat gluten or egg protein. The disadvantage of wheat gluten is that is a potential allergen. Further, the disadvantage of egg protein is that the produced food item is not vegan, and a potential allergen.

In general there is a need for alternative binders or gelling agents.

### SUMMARY

The invention provides:
a method for preparing a non-animal protein food product or a cultured meat food product comprising
   adding a thermolabile glucoamylase protein preparation to an ingredient used in the preparation of said food product and processing the obtained glucoamylase protein preparation and ingredient composition into a non-animal protein food product or a cultured meat food product;
a food product obtainable by one of the herein described methods;
meat or fish alternative food product comprising non-animal protein or cultured meat and a thermolabile glucoamylase protein preparation;
meat or fish alternative which comprises texturized vegetable protein, non-animal protein, water, flavour and a binder system, wherein the binder system comprises a thermolabile glucoamylase protein preparation; and
use of a thermolabile glucoamylase protein preparation for obtaining binding in a food product or for at least partly replacing a binder in a food product.

### DETAILED DESCRIPTION

Food binders are food additives that are added to food products for the purpose of improving texture via thickening or binding the ingredients together. Food binders play an important role in the production of food by for example improving texture, juiciness and/or increasing volume.

Examples of widely used food binders are eggs, wheat flour, oatmeal, rice, milk, gelatine, guar gum, xanthan gum, (potato) starch or methyl cellulose.

There is a need in the art for alternative binders or alternative gelling agents. The present application addresses this need.

As disclosed herein within the experimental part, a thermolabile glucoamylase protein preparation can surprisingly be used as a binder or as a gelling agent, preferably as a gelling agent.

Throughout the present specification and the accompanying claims, the words “comprise”, “include” and “having” and variations such as “comprises”, “comprising”, “includes” and “including” are to be interpreted inclusively. That is, these words are intended to convey the possible inclusion of other elements or integers not specifically recited, where the context allows.

The articles “a” and “an” are used herein to refer to one or to more than one (i.e. to one or at least one) of the grammatical object of the article. By way of example, “an element” may mean one element or more than one element.

In one of its aspects, the invention provides a method for preparing a non-animal protein food product or a cultured meat food product comprising
adding a thermolabile glucoamylase protein preparation to an ingredient used in the preparation of said food product and processing the obtained glucoamylase protein preparation and ingredient composition into a non-animal protein food product or a cultured meat food product.

Alternatively, this aspect of the invention can be worded as:
a method for preparing a non-animal protein food product comprising adding a thermolabile glucoamylase protein preparation to an ingredient used in the preparation of said food product and processing the obtained glucoamylase protein preparation and ingredient composition into a non-animal protein food product, or
a method for preparing a cultured meat food product comprising adding a thermolabile glucoamylase protein preparation to an ingredient used in the preparation of said food product and processing the obtained glucoamylase protein preparation and ingredient composition into a cultured meat food product.

The term “non-animal protein food product” as used herein refers to a food product which does not comprise animal protein.

The term “cultured meat food product” as used herein refers to a food product which is prepared from cultured meat. Cultured meat is a meat produced by in vitro cell cultures of animal cells. It is a form of cellular agriculture. Cultured meat is produced using tissue engineering techniques traditionally used in regenerative medicines.

The term “an ingredient used in the preparation” as used herein covers any ingredient which is used in the preparation of a non-animal protein food product or a cultured meat food product. A suitable ingredient depends on the final non-animal protein food product or a cultured meat food product. As a non-limiting example, a meat alternative product such as a hamburger patty, sausages, or nuggets is described herein in more detail. Preferably, said meat alternative product is a hamburger. Typical ingredients of such a meat alternative are texturized vegetable protein, (non-animal) protein, oil and/or fat, water and a flavour system. The thermolabile glucoamylase protein preparation may be added to any of these ingredients, for example to the texturized vegetable protein or to the protein or to the oil and/or fat or to the water or to the flavour system. In one of the embodiments, all (or part) of the ingredients of the non-animal protein food product or a cultured meat food product are added to each other (and optionally mixed) and in a next step the glucoamylase protein preparation is added. In yet another embodiment, all ingredients of the non-animal protein food product or a cultured meat food product and the thermolabile glucoamylase protein preparation are added to each other at approximately the same time. In one of the embodiments the thermolabile glucoamylase protein preparation may be added during the preparation of the texturized vegetable protein. Preferably, the term “an ingredient used in the preparation” does not comprise starch.

In case the meat or fish alternative is based on a High Moisture Extrudate (HME), the thermolabile glucoamylase protein preparation may be added during the preparation of the HME, or alternatively to the HME or to any of the other usual ingredients (e.g. oils, fats, proteins, vitamins, minerals) of the meat or fish alternative. Preferably, said meat or fish alternative does not comprise starch.

In case the meat or fish alternative is based on Shear Cell Technology, the thermolabile glucoamylase protein preparation may be added during the preparation of the protein slab, or alternatively to the protein slab or to any of the other usual ingredients (e.g. oils, fats, proteins, vitamins, minerals) of the meat or fish alternative. Preferably, said meat or fish alternative does not comprise starch.

In case the non-animal protein food product is a vegan cheese (also referred to as plant-based dairy cheese alternative), the term “an ingredient used in the preparation” typically includes gums and/or a non-animal protein source as binder, vegetable fats or oil and other ingredients (salt, calcium, acid, preservatives, flavourings, color). Preferably, said vegan cheese does not comprise starch.

In case the non-animal protein food product is a plant-based milk or yogurt (also referred to as plant-based dairy alternative), the term “an ingredient used in the preparation” typically includes plant-based milks like soy, oat, coconut, rice, almond, and lactic acid bacteria (in yoghurt) and other ingredients (gums, vitamins, flavourings or sugars etc). Preferably, said plant-based milk or yogurt does not comprise starch.

In case the non-animal protein food product is a vegan omelet or other egg-replacer, the term “an ingredient used in the preparation” typically includes plant-based protein, binders like methylcellulose, and other ingredients (flavourings, color). Preferably, methylcellulose is replaced by glucoamylase protein.

The step of adding a thermolabile glucoamylase protein preparation to an ingredient used in the preparation of a non-animal protein food product or a cultured meat food product, generally comprises
combining a thermolabile glucoamylase protein with plant protein, microbial protein or algal protein
combining a thermolabile glucoamylase protein with plant protein
combining a thermolabile glucoamylase protein with microbial protein
combining a thermolabile glucoamylase protein with fungal protein
combining a thermolabile glucoamylase protein with bacterial protein
combining a thermolabile glucoamylase protein with algal protein
combining a thermolabile glucoamylase protein preparation with texturized vegetable protein
combining a thermolabile glucoamylase protein preparation with a plant-based milk
preparing of a binder phase wherein said thermolabile glucoamylase protein preparation is combined with oil or fat and water
preparing of a binder phase wherein said thermolabile glucoamylase protein preparation is combined with oil or fat, water and a hydrocolloid
combining a thermolabile glucoamylase protein preparation with other dry ingredients
combining a thermolabile glucoamylase protein preparation with other dry ingredients, oil and water
combining a thermolabile glucoamylase protein preparation with seasoning (such as, but not limited to, salt and/or pepper), or
combining a thermolabile glucoamylase protein preparation with water and a yeast extract

Preferably, the term “an ingredient used in the preparation” as used herein excludes starch as an ingredient.

The phrase “processing the obtained glucoamylase and ingredient composition into a non-animal protein food product or a cultured meat food product” comprises well know steps such as mixing, kneading, fermenting, cooking, frying, baking, freezing, extrusion, shear cell technology and many others.

The term “obtained glucoamylase protein preparation and ingredient composition” refers to the composition which is obtained when a thermolabile glucoamylase protein preparation is added to an ingredient used in the preparation of any of said food products.

Glucoamylase is an enzyme which is produced by many micro-organisms but is for industrial uses typically obtained from Aspergillus or Rhizopus species, like Aspergillus niger, Aspergillus awamori and Rhizopus oryzae. Most preferably glucoamylase is produced by Aspergillus niger. The glucoamylase enzyme is also often named amyloglucosidase or EC 3.2.1.3-glucan 1,4-alpha-glucosidase and is currently used for saccharification of starch. Its main applicability is in brewing, bioethanol production, baking and in the starch industry for production of glucose syrup. The herein described method does not rely on the enzymatic activity of glucoamylase. As disclosed herein in the experimental part glucoamylase protein is very suitable as a binder or gelling agent and hence disclosed herein is a method for preparing a non-animal protein food product or a cultured meat food product comprising
adding a thermolabile glucoamylase protein preparation to an ingredient used in the preparation of said food product and processing the obtained glucoamylase protein preparation and ingredient composition into a non-animal protein food product or a cultured meat food product, wherein said glucoamylase protein preparation provides binding or gelling properties to the food product when said glucoamylase protein preparation and ingredient composition or said food product is heated to at least 65 degrees Celsius.

Preferably disclosed herein is a method for preparing a non-animal protein food product or a cultured meat food product comprising
adding a thermolabile glucoamylase protein preparation to an ingredient used in the preparation of said food product and processing the obtained glucoamylase protein preparation and ingredient composition into a non-animal protein food product or a cultured meat food product, wherein said glucoamylase protein preparation provides gelling properties to the food product when said glucoamylase protein preparation and ingredient composition or said food product is heated to at least 65 degrees Celsius.

The binding properties are obtained by gelling of the thermolabile glucoamylase protein. Preferably the gelling results in the formation of an irreversible gel.

Preferably, the thermolabile glucoamylase protein preparation is used to replace a conventional binder such as methylcellulose and hence alternatively phrased, the invention provides a method for reducing (preferably completely reducing) methylcellulose in preparing a non-animal protein food product or a cultured meat food product comprising
adding a thermolabile glucoamylase protein preparation to an ingredient used in the preparation of said food product and processing the obtained glucoamylase protein preparation and ingredient composition into a non-animal protein food product or a cultured meat food product.
The term “thermolabile glucoamylase” refers to a glucoamylase which is enzymatically active at relative lower temperatures (as opposed to a thermostable glucoamylase). Enzymatic activity of a glucoamylase is for example determined by incubating the enzyme on a suitable substrate such as maltose. Whether or not the glucoamylase is thermostable or thermolabile is determined by testing the enzymatic activity on the substrate at different temperatures. A thermolabile glucoamylase is defined herein as a glucoamylase which is enzymatically active at a temperature below 70 degrees Celsius (i.e. a thermolabile glucoamylase is not or hardly not active at a temperature above 80 degrees Celsius).

In the aspect of the invention which is outlined above (i.e. a method for preparing a non-animal protein food product or a cultured meat food product or a method for reducing (preferably completely reducing) methylcellulose in preparing a non-animal protein food product or a cultured meat food product) said method can comprise a heating step, i.e. the above described methods further comprise a step of heating to at least 65 degrees Celsius. As disclosed herein within the experimental part, heating to at least 65 degrees Celsius results in gelling of thermolabile glucoamylase protein. A suitable upper limit for heating depends on the final product as well as on the used method for heating (for example with or without pressure) and can easily be determined by the skilled person. I.e the gelling as described herein refers to gelling after heating, preferably heating to at least 65 degrees Celsius.

The term “glucoamylase” is explained above. The term “thermolabile glucoamylase” is also explained above. Glucoamylase is typically sold as a glucoamylase protein preparation which does not only comprise the glucoamylase protein but also other components such as a salt and/or a preservative. Glucoamylase preparations have been described for and isolated from a large variety or organisms, including micro-organisms. Glucoamylase is often an extracellular component of bacteria or fungi, like Bacillus or Aspergillus. Glucoamylase from Aspergillus and especially Aspergillus niger and Aspergillus awamori, but also protein engineered or evolved variants thereof, are often being used in industrial practice. Other fungal glucoamylases such as those of the genera Rhizopus, Penicillium or Talaromyces may also be used. Typically, a glucoamylase protein preparation also comprises other components such as other proteins, which are derived from/expressed by the micro-organism. Hence, the thermolabile glucoamylase protein preparation may comprise other components. For example, the preparation of intracellular glucoamylase requires the disruption of the cells to release the glucoamylase protein. At the same time, other cytoplasmic proteins/enzymes are released. For the present invention, crude glucoamylase protein preparations can be used.

Examples of a suitable thermolabile glucoamylase protein preparation are a glucoamylase protein preparation which comprises
a native glucoamylase protein
a fungal glucoamylase protein
an Aspergillus glucoamylase protein
an Aspergillus niger glucoamylase protein
a classical (non-GMO) glucoamylase protein,
an extracellularly produced glucoamylase protein, and/or
any combination thereof.

Preferably, the thermolabile glucoamylase protein preparation comprises a glucoamylase protein having its pH optimum in the range of pH 4 to 5 or said preparation comprises an Aspergillus glucoamylase protein and more preferably the glucoamylase protein is Aspergillus glucoamylase which is endogenously expressed by Aspergillus. The expression of the glucoamylase protein in Aspergillus is preferably enhanced by classical strain improvement. Most preferred is a glucoamylase protein preparation which glucoamylase is endogenously expressed by Aspergillus and which expression is enhanced by classical strain improvement.

Suitable examples of a thermolabile glucoamylase protein preparation are Amigase Mega L, Bakezyme AG800, Hazyme DCL products (DSM Food & Beverage), Sunson GA130L (Sunson), Glucoamylase GA-300S (Hunan NHY), Sunbake AMG NG (Suntaq), Distillase, Diazyme X4 (IFF) or AMG (Novozymes).

As described above and as shown in the experimental part herein, other thermolabile glucoamylase protein preparations can be used as well. Even more preferably, the used thermolabile glucoamylase protein preparation does not comprise glycerol or any compound (such as maltodextrin) which is typically used in spray drying of enzymes.

As described above, the herein described method does not rely on the enzymatic activity of a thermolabile glucoamylase and hence the enzymatic activity of the glucoamylase may be inhibited by a suitable inhibitor without losing its functionality as binder.

The phrase “native glucoamylase protein” refers to a non-denatured glucoamylase protein which has enzymatic activity.

The thermolabile glucoamylase is used in the present invention not for its enzymatic activity but as a (n) (further) ingredient in a non-animal protein food product or a cultured meat food product and more in specific as an ingredient which forms a gel after heating.

The thermolabile glucoamylase is added in a sufficient amount as a gelling agent such that after heating the glucoamylase protein forms a gel. The amount of glucoamylase protein preparation which is added to get the desired binding effect can easily be determined by the skilled person. Guidance can be found in the experimental part herein. Based on total protein (present in the used glucoamylase protein preparation) a dosage of 0.05 to 20% or 0.05 to 10% is sufficient to result in binding or gelling. More preferably, at least 0.1% of total protein (present in the used glucoamylase protein preparation) is used and a suitable range is at least 0.1% to 20%. Most preferably, at least 0.5% of total protein (present in the used glucoamylase protein preparation) is used and hence the most preferred range is at least 0.5% to 20%. The presented amounts of glucoamylase protein preparation are relative to the mass of the food product. Not all the proteins in a glucoamylase protein preparation are glucoamylase protein but—as glucoamylase production strains are designed to produce as much glucoamylase protein as possible—an amount of 50% glucoamylase protein (based on total protein in the glucoamylase protein preparation) is typical. Based on glucoamylase protein (present in the used glucoamylase protein preparation) a dosage of 0.025 to 10% or 0.025 to 5% is sufficient to result in binding. More preferably, at least 0.05% of glucoamylase protein (present in the used glucoamylase protein preparation) is used and a suitable range is at least 0.05% to 10%. Most preferably, at least 0.25% of glucoamylase protein (present in the used glucoamylase protein preparation) is used and a suitable range is at least 0.25% to 10%. The presented amounts of glucoamylase protein is relative to the mass of the food product. The skilled person is capable of determining the amount of glucoamylase protein in a glucoamylase protein preparation by using well known methods. For example, HP-SEC or SDS-PAGE analysis can be used to estimate the relative amount of glucoamylase protein in comparison to other proteins in the glucoamylase protein preparation.

The term “non-animal protein food product” as used herein refers to a food product which does not comprise any animal protein. The non-animal protein food product comprises proteins from other sources. The non-animal protein is for example plant protein, microbial protein or algal protein. Suitable examples of a microbial protein are fungal protein or bacterial protein. I.e. the non-animal protein food product is for example a plant protein food product, a microbial protein food product, an algal protein food product, a fungal protein food product or a bacterial protein food product

The term “plant protein food product” as used herein refers to a “food product comprising a plant protein” (alternatively referred to as “plant protein comprising food product”; the phrases are used interchangeably herein) which refers to a food product which comprises at least 10% (based on all proteins present in said food product) plant protein. Preferably, said plant-based food product comprises at least 20%, 30%, 40%, 50%, 60%, 70%, 80% or 90% (based on all proteins present in said food product) plant protein. Most preferably, said plant protein food product comprises only or exclusively (i.e. 100% based on all proteins present in said food product) plant protein and no animal-derived at all. As used herein, the term “plant protein” refers to any protein from plant origin. Preferably, the plant protein is a protein from grains, pseudocereals, legumes, nuts, seeds or other sources such as coconut, potato, canola or tiger nut.

Examples of suitable grains are barley, fonio, maize, millet, oat, rye, sorghum, teff, triticale, spelt, rice or wheat.

Examples of suitable pseudograins are amaranth, buckwheat or quinoa.

Examples of suitable legumes are lupin, pea, chickpea, beans (preferably faba beans), duckweed, potato, peanut or soy.

Examples of suitable nuts are almond, brazil, cashew, hazelnut, macadamia, pecan, pistachio or walnut.

Examples of suitable seeds are canola seed, chia seed, flax seed, hemp seed, pumpkin seed, sesame seed or sunflower seed.

The term “microbial protein food product” as used herein refers to a “food product comprising a microbial protein” (alternatively referred to as “microbial protein comprising food product”; the phrases are used interchangeably herein) which refers to a food product which comprises at least 10% (based on all proteins present in said food product) microbial protein. Preferably, said microbial protein food product comprises at least 20%, 30%, 40%, 50%, 60%, 70%, 80% or 90% (based on all proteins present in said food product) microbial protein. Most preferably, said microbial protein food product comprises only or exclusively (i.e. 100% based on all proteins present in said food product) microbial protein and no animal-derived at all. Preferably, the microbial protein is a fungal protein or a bacterial protein and hence a fungal protein food product or a bacterial protein food product is obtained.

The term “fungal protein food product” as used herein refers to a “food product comprising a fungal protein” (alternatively referred to as “fungal protein comprising food product”; the phrases are used interchangeably herein) which refers to a food product which comprises at least 10% (based on all proteins present in said food product) fungal protein. Preferably, said fungal protein food product comprises at least 20%, 30%, 40%, 50%, 60%, 70%, 80% or 90% (based on all proteins present in said food product) fungal protein. Most preferably, said fungal protein food product comprises only or exclusively (i.e. 100% based on all proteins present in said food product) fungal protein and no animal-derived at all. An alternative term for a fungal protein is a mycoprotein Suitable examples of a mycoprotein are proteins derived from, for example, from Fusarium venenatum. Such a product is commercially available under the name Quorn. Other suitable sources of mycoprotein are Neurospora crassa, Thermomucor indicae-seudaticae, Lentinula edodes (Shiitake), Pleurotus ostreatus (Oyster mushroom), Rhizopus, Fusarium oxysporum, Fusarium novum-yellowstonensis or Aspergillus oryzae (Koji).

The term “bacterial protein food product” as used herein refers to a “food product comprising a bacterial protein” (alternatively referred to as “bacterial protein comprising food product”; the phrases are used interchangeably herein) which refers to a food product which comprises at least 10% (based on all proteins present in said food product) bacterial protein. Preferably, said bacterial protein food product comprises at least 20%, 30%, 40%, 50%, 60%, 70%, 80% or 90% (based on all proteins present in said food product) bacterial protein. Most preferably, said bacterial protein food product comprises only or exclusively (i.e. 100% based on all proteins present in said food product) bacterial protein and no animal-derived at all. Suitable examples of bacterial proteins can be obtained from Xanthobacter tagetidis or Cupriavidus necator

The term “algal protein food product” as used herein refers to a “food product comprising an algal protein” (alternatively referred to as “algal protein comprising food product”; the phrases are used interchangeably herein) which refers to a food product which comprises at least 10% (based on all proteins present in said food product) algal protein. Preferably, said algal protein food product comprises at least 20%, 30%, 40%, 50%, 60%, 70%, 80% or 90% (based on all proteins present in said food product) algal protein. Most preferably, said algal protein food product comprises only or exclusively (i.e. 100% based on all proteins present in said food product) algal protein and no animal-derived at all. Suitable examples of algal proteins are Chlamydomonas, Spirulina (commercially produced by Damhert), Euglena gracilis, Odontella, Saccharina (commercially produced by Viva Maris) or Chlorella (commercially produced by Alver).

The herein described non-animal protein food product (for example microbial protein food product, fungal protein food product, bacterial protein food product or algal protein food product) or the herein described cultured meat food product is typically sold as a meat-alternative food product or a fish alternative food product. Hence, also described herein is a method for preparing meat alternative food product or a fish alternative food product comprising
adding a thermolabile glucoamylase protein preparation to an ingredient used in the preparation of said food product and processing the obtained glucoamylase protein preparation and ingredient composition into a meat alternative food product or a fish alternative food product.

Alternatively, this aspect of the invention can be worded as:
a method for preparing a meat alternative food product comprising adding a thermolabile glucoamylase protein preparation to an ingredient used in the preparation of said food product and processing the obtained glucoamylase protein preparation and ingredient composition into a meat alternative food product, or
a method for preparing a fish alternative food product comprising adding a thermolabile glucoamylase protein preparation to an ingredient used in the preparation of said food product and processing the obtained glucoamylase protein preparation and ingredient composition into a fish alternative food product.

The term meat- or fish-alternative, meat or fish analogue product, or meat or fish substitute, as used in the present context means a product that does not comprise animal or fish protein and thus is suitable to be used as a vegetarian or vegan meat- or fish-alternative and has an appearance mimicking an animal meat- or fish-based product. Meat- or fish-alternatives may be in the form of patties, nuggets, sausages, cold cut, spreads, sticks or any other form.

Alternatively, any of the above-described methods (i.e. a method for preparing a non-animal protein food product or a cultured meat food product or a method for reducing (preferably completely reducing) methylcellulose in preparing a non-animal protein food product or a cultured meat food product) is used to produce a non-animal protein food product such as a cheese, milk, egg or yoghurt like food product, for example a plant-based cheese (alternatively referred to as vegan cheese), a plant-based yoghurt, or plant-based milk alternative, plant-based ice-cream, etc.

Hence, also described herein is a method for preparing a non-animal protein food product comprising
adding a thermolabile glucoamylase protein preparation to an ingredient used in the preparation of said food product and processing the obtained glucoamylase protein preparation and ingredient composition into a non-animal protein food product and wherein said food product is a plant-based cheese (alternatively referred to as vegan cheese), a plant-based yoghurt, plant-based milk alternative or plant-based ice-cream.

The thermolabile glucoamylase protein preparation used in any of the herein described methods is added to obtain binding properties and is not added to process starch/dextrins or maltose into glucose.

In yet another aspect, the invention further provides a food product obtainable by any of the methods described herein and hence the food products may be a meat-alternative or a fish-alternative or a non-animal protein food product or a cultured meat food product or any of the above-mentioned food products. Such a food product differs from other food products in that it comprises thermolabile glucoamylase. Prior art uses of a glucoamylase are limited to the use in the baking industry, in brewing, starch processing, high-fructose corn syrup production, bioethanol production and the like. Herein, glucoamylase is used as a binder or as a gelling agent, preferably as a binder or gelling agent to replace methylcellulose. The thermolabile glucoamylase is added in much higher amounts when compared to its traditional use in the saccharification in different industries wherein typically ppm amounts of glucoamylase protein is added.

The invention further provides a meat or fish alternative food product comprising non-animal protein or cultured meat and a thermolabile glucoamylase protein preparation. The explanation provided above for the different features in respect of the method claims equally apply to this part of the invention.

An additional component of such a meat or fish alternative food product is non-animal fat.

The invention also provides meat or fish alternative which comprises texturized vegetable protein, non-animal protein, water, flavour and a binder system, wherein the binder system comprises a thermolabile glucoamylase protein preparation. In one embodiment the thermolabile glucoamylase protein preparation can (also) be present in the texturized vegetable protein.

Preferably the present meat- or fish-alternative comprises texturized vegetable protein (TVP). Preferably the texturized vegetable protein is an extruded vegetable protein product. This can cause a change in the structure of the protein which results in a fibrous, spongy matrix, similar in texture to meat. The textured vegetable protein can be rehydrated or dehydrated. Preferably, the texturized vegetable protein is selected from soybean protein, pea protein, lentil protein, lupin bean protein, wheat gluten, rapeseed protein, fava bean protein or a combination thereof. Given that soy is an allergen, it is preferred that the present texturized vegetable protein is soy free. Preferably the present meat- or fish-alternative is soy free.

Preferably, the present meat- or fish-alternative comprises texturized vegetable protein in an amount from 5 to 30% (w/w), preferably an amount of 6 to 25% (w/w), preferably 8 to 20% (w/w), preferably 10 to 15% (w/w) of the meat- or fish-alternative.

Preferably, the present meat- or fish-alternative comprises texturized vegetable protein with a protein amount from 50 to 99% (w/w), preferably an amount of 55 to 90% (w/w), preferably 60 to 85% (w/w) of the texturized vegetable protein.

Preferably, the present texturized vegetable protein is hydrated towards an amount of water of more than 10% (w/w) of the texturized vegetable protein, preferably an amount of water from 20 to 80% (w/w) of the texturized vegetable protein, preferably an amount of water from 30 to 70% (w/w) of the texturized vegetable protein.

The term “binder” or “binding agent” as used herein relates to a substance for holding together particles and/or fibres in a cohesive mass. It is an edible substance that in the final product is used to trap components of the foodstuff with a matrix for the purpose of forming a cohesive product and/or for thickening the product. Binding agents of the invention may contribute to a smoother product texture, add body to a product, help retain moisture and/or assist in maintaining cohesive product shape; for example by aiding particles to agglomerate. The amount of glucoamylase protein preparation which needs to be added to get the desired binding effect can easily be determined by the skilled person. Guidance can be found in the experimental part herein. Based on total protein (present in the used glucoamylase protein preparation) 0.05 to 10% is sufficient to result in binding.

Preferably the present meat- or fish-alternative does not comprise methyl cellulose and/or wheat gluten.

In a preferred embodiment, the present meat- or fish-alternative further comprises a nutrient, preferably wherein the nutrient comprises both vitamins and minerals, preferably vitamins chosen from the group consisting of B2, B3, B6 and B12, preferably minerals chosen from the group consisting of iron, selenium and zinc. The term “nutrient” as used herein relates to a substance that provide nutritional value to the present meat- or fish-alternative, such as vitamins, minerals, trace elements and antioxidants for example. The advantage of adding these nutrients is that the present meat- or fish-alternative more closely resembles the nutritional value of a real meat hamburger, without introducing off flavors to the meat- or fish-alternative.

In an embodiment, the present meat- or fish-alternative further comprises a vegetable oil and/or a vegetable fat. The vegetable oil and/or fat can be an algal oil, a fungal oil, corn oil, olive oil, soy oil, peanut oil, walnut oil, almond oil, sesame oil, cottonseed oil, rapeseed oil, canola oil, safflower oil, sunflower oil, flax seed oil, palm oil, palm kernel oil, coconut oil, babassu oil, shea butter, mango butter, cocoa butter, wheat germ oil, borage oil, black currant oil, sea-buckhorn oil, macadamia oil, saw palmetto oil, conjugated linoleic oil, arachidonic acid enriched oil, docosahexaenoic acid (DHA) enriched oil, eicosapentaenoic acid (EPA) enriched oil, palm stearic acid, sea-buckhorn berry oil, macadamia oil, saw palmetto oil, or rice bran oil; or margarine or other hydrogenated fats. In some embodiments, for example, the oil is algal oil. In a preferred embodiment, the present plant oil is sunflower oil and/or the present plant fat is coconut fat.

Preferably, the amount of vegetable oil is within the range from 2 to 20% (w/w) of the meat- or fish-alternative, such as from 5 to 15% (w/w) or from 7 to 12% (w/w). Preferably, the amount of vegetable fat is within the range from 0.5 to 5% (w/w) of the meat- or fish-alternative, such as from 1 to 3% (w/w) of the meat- or fish-alternative.

Preferably, the meat-alternative is a hamburger patty, a nugget, a minced meat, a meat ball or a sausage. Preferably, the meat-alternative does not comprise starch. Most preferred is a hamburger patty. Preferably, the hamburger does not comprise starch.

Preferably, the fish-alternative is a batter fish alternative, a fish burger alternative, a smoked fish alternative, a fish salad alternative or a fish ball alternative. Preferably, the fish-alternative does not comprise starch.

Preferably the meat- or fish-alternative comprises a protein or protein isolate or protein concentrate. Examples of proteins can be barley, fonio, maize, millet, oat, rye, sorghum, teff, triticale, spelt, rice, wheat, amaranth, buckwheat, quinoa, lupin, pea, chickpea, beans (preferably faba beans), duckweed, potato, lentil, peanut, soy, almond, brazil, cashew, hazelnut, macadamia, pecan, pistachio, walnut, canola, chia, flax, hemp, pumpkin, sesame, sunflower, mycoprotein (e.g. Quorn), mushroom or algae (e.g. Chlamydomonas, Spirulina, Euglena, Odontella, Saccharina, Chlorella). More preferably, the present meat or fish-alternative product comprises 0.001 to 20% (w/w) of a protein.

Preferably the meat- or fish-alternative comprises a flavour or flavour agent, or flavour precursor. Examples of flavours can be yeast extracts or process flavours. More preferably the present meat or fish-alternative product comprises 0.001 to 5% (w/w) of a flavour.

In an embodiment the present meat- or fish-alternative comprises a flavour modifier or a flavoring with modifying properties. More preferably the present meat- or fish-alternative comprises 0.001 to 1% (w/w) of a flavour modifier or a flavouring with modifying properties.

Preferably, the present meat- or fish-alternative comprises salt, preferable NaCl. The amount of salt is preferably within the range of 0.001 to 5% (w/w) of the present meat- or fish-alternative.

In a preferred embodiment, the present meat- or fish-alternative comprises a colorant, preferably within the range of 0.01 to 10 wt. %, more preferably 0.1 to 5 w. %, most preferably 0.2 to 2 wt. % of the meat- or fish-alternative. In a preferred embodiment, the present colorant comprises or is beet root or beet root powder. The advantage of using beet root is that a meaty like color is provided to the product, without introducing off flavors to the product. The present colorant can also be or comprise a carotenoid. Preferably, the carotenoid is chosen from the group consisting of α- or ß-carotene, 8′-apo-β-carotenal, 8′-apo-ß-carotenoic acid esters such as the ethyl ester, bixin, capsanthin, capsorubin, rhodoxanthin, canthaxanthin, astaxanthin, astaxanthin esters, lycopene, lutein, zeaxanthin or crocetin and their derivatives.

In an embodiment the present meat- or fish-alternative comprises a heme, a heme protein, a heme containing protein or a (macro) molecule with complexed iron. More preferably the present meat- or fish-alternative comprises 0.001 to 5% (w/w) of a heme, a heme protein, a heme containing protein or a (macro) molecule with complexed iron.

In an embodiment, the present meat- or fish-alternative comprises an amount of water within the range of 50 to 80% (w/w), preferably 55 to 70% (w/w).

In yet another aspect, the invention also provides use of a thermolabile glucoamylase protein preparation for obtaining binding or gelling in a food product or for at least partly replacing a binder in a food product. A binder can be part of a binder system. For example, the binder methylcellulose can be part of a binder system which additionally comprises oil, gums and water. Preferably, at least 10% or at least 20, 30 or 40% and more preferably at least 50, 60 or 70% and even more preferably at least 80, 90 or 95% of the binder is replaced and most preferred 100% of the binder is replaced by said glucoamylase. An example of a binder which can be at least partly replaced is methylcellulose. Described herein are use of a thermolabile glucoamylase protein preparation for at least reducing the amount of methylcellulose in a food product. Preferably, the food product is a meat-alternative food product or a fish-alternative food product or any of the above-described food products. More preferably, methylcellulose is completely replaced as a binder. Preferably, said food product is a meat alternative and most preferred is a hamburger alternative. The explanation provided above for the different features in respect of the method and product claims equally apply to this part of the invention.

The invention further provides use of a thermolabile glucoamylase protein preparation as a gelling agent. Preferably use of a thermolabile glucoamylase protein preparation as a gelling agent in a non-animal protein food product or a cultured meat food product.

The invention will be explained in more detail in the following example, which are not limiting the invention.

### EXAMPLES

### Rheology Measurements

Dynamic oscillatory rheology was performed using an Anton Paar Physica rheometer MCR302 with a cup and bob geometry (CC27), and a program as displayed in table 1. During the measurements rheological data was obtained that is expressed as the complex modulus G* [Pa] and the phase angle [°]. The measurement was performed by filling the cup with 17-20 ml protein dispersion. To prevent samples from drying out during the experiment, the sample in the cup was covered with a thin layer of sunflower oil. The test program in detail: a temperature sweep was conducted, the sample was heated and then cooled over a temperature range of 25-95° C. with steps 2° C. per minute followed by a 10 minutes holding time at the final temperature of 95° C. A constant frequency of 1 Hz and strain of 0.1% were applied to collect data during the heat set and the cooling down phase. Rheological data were collected at 30 seconds intervals. After the gel was cooled, it was held at 25° C. for 10 minutes now with a frequency of 0.1 Hz and strain of 0.1%. During this step, rheological data were collected at 1-minute intervals. Subsequently, a strain sweep was performed on the heat-set protein gel, using a constant frequency of 0.1 Hz and increasing strain from 0.1 to 100% at a constant temperature of 25° C.

TABLE 1
Program settings of rheometer

| | | | | | | | Total time | Heating |
|---|---|---|---|---|---|---|---|---|
| | Temperature | | Frequency | Strain γ | # of | Time per | of the | rate |
| Step | [° C.] | | [Hz] | [%] | points | point [s] | phase [s] | [° C./min] |
| 1 | 25 → 95° | C. | 1 | 0.1 | 70 | 30 | 2100 | 2 |
| 2 | 95° | C. | | | 20 | 30 | 600 | 0 |
| 3 | 95 → 25° | C. | | | 70 | 30 | 2100 | −2 |
| 4 | 25° | C. | 0.1 | | 10 | 60 | 600 | 0 |
| 5 | 25° | C. | | 0.1 → 100 | 31 (10 | Set by | Set by | 0 |

| | | | |
|---|---|---|---|
| logarithmically | points/decade) | rheometer | rheometer |

### Texture Profile Analysis (TPA)

Texture profile analysis was performed on a Texture analyzer (TA.TA.Xtplus, Stable Microsystems Ltd Surrey, UK) provided with the software “Exponent”. A cylindric probe with 45 mm diameter and a load cell of 5 kg was used. Double compression test to 30% deformation, at 3 mm/sec test speed with a 2 sec delay between first and second compression was performed. All samples were measured in 5-fold, and averages and standard deviation was calculated. Hardness, cohesiveness, springiness and resilience were calculated by the instrument software. Gumminess (hardness*cohesiveness) and Chewiness (hardness*cohesiveness*springiness) can be calculated herefrom.

### Example 1: Heat-Set of Glucoamylase Protein Preparations

Different glucoamylase protein preparations were tested for heat-setting behavior by heating a 1 ml sample in a 2 ml Eppendorf tube for 5 minutes at either 60, 70 or 80° C. Liquid enzymes were directly tested, while enzymes in powder form were first resuspended at 15% (w/V) in cold 50 mM phosphate-buffer (pH 7.0) and solids were removed by centrifugation before the enzymes were tested. After heating the development of a self-supporting gel was recorded by inverting the tubes. The tested glucoamylase protein preparations were:

TABLE 2
Heat-gelation behaviour of commercial glucoamylases.

| | | | 60° | 70° | 80° |
|---|---|---|---|---|---|
| Enzyme | Supplier | Form | C. | C. | C. |
| Amigase Mega | DSM | liquid | ± | + | + |
| Hazyme DCL | DSM | liquid | − | + | + |
| Bakezyme AG800 | DSM | powder | − | + | + |
| Glucoamylase HY01 | Hunan NHY | liquid | ± | + | + |
| Glucoamylase GA-300S | Hunan NHY | powder | − | + | + |
| Sunbake AMG NG | Suntaq | powder | − | + | + |

− means no gelation,
± means weak gel formation,
+ means that a self-sustaining gel was formed

After testing an enzyme collection of globular proteins, many of them did not show gelation after incubation for 10 minutes at 80 degrees C. Examples of non-heat-gelling enzymes are:
Bacterial amylase (Bacillus amyloliquefaciens)
Arabinofuranosidase (Aspergillus niger)
Xylanase (Aspergillus niger)
Xylanase (Trichoderma reesei)
Protease (Aspergillus oryzae)
Lipase (Rhizopus oryzae)
Protease (Rhizomucor miehei)
Beta-glucosidase (Aspergillus niger)
Invertase (Saccharomyces cerevisiae)

Testing at lower temperatures (60 or 70 degrees Celsius) resulted in a larger set of enzymes (globular proteins) which did not show gelation after incubation for 10 minutes at 60 or 70 degrees Celsius.

### Example 2: Gel Strength and Elastic Behavior

A 11.1% (w/V) solution of egg-white powder (Sanovo) was made in water by gently stirring until a homogeneous suspension is formed. The suspension contains a protein content of ~10% (w/v). Amigase Mega (a 30% protein solution) was diluted 3 times with water to obtain a ~10% (w/v) protein solution. Sunbake AMG NG (a 60% protein granulate) was suspended at 10% (w/V) in cold water and solids were removed by centrifugation. Similarly, 2.5% (w/V) solutions of egg white and Amigase Mega were made.

Heat-gelation was performed in the Rheometer as described above. Heat gelation onset temperature of the glucoamylase samples was very similar to that of egg white and occurred at 65-70° C. at the high protein concentration and at 70-75° C. at the low protein concentration (FIGS. 1A and 2A). Maximum modulus after heating of the glucoamylase was comparable to egg white at the low concentration, but slightly lower for the 10% solutions. Gel strength was increased further during the cooling to 25° C. The complex shear modulus of the cooled gel was hardly affected by the strain applied (FIGS. 1B and 2B) indicating that the glucoamylase gels show a highly elastic behavior and do not break when applying strain.

### Example 3: Use of a Glucoamylase Amigase Mega in Vegan Hamburgers

The ingredients as shown in table 3 are used in preparation of the hamburgers in the following order. First caramelized sugar and beetroot powder are added to water 1. This solution is used to hydrate TVP TU-crumble caramel 180 (ADM) and left for hydration for at least 45 minutes at room temperature. During those 45 minutes, the product is mixed for 15 seconds every 15 minutes, using a Hobart kitchen machine.

To prepare the binder phase, first the solid ingredients (texturants 1; MC, Gellan, Pectin) and oil are mixed in a Magimix for 60 seconds. Then the Aglufiber (texturant 2) and protein ingredients are stirred into the water 2. This dispersion is mixed in a Magimix for 5 minutes. The final dispersion or ice-water (for MC burger) is slowly added while mixing under high shear. This phase is mixed for an extra 5 minutes. At this stage the paste like emulsion is ready and subsequently mixed with the hydrated TVP until homogenous appearance of the dough. Finally, the dry parts soy protein isolate, flavours and salt are mixed in the dough, followed by the frozen coconut fat chunks. Added protein concentration of the tested samples in the final burger is calculated to be 3% (w/w) and substitutes the 3% soy protein isolate (SPI) in the control burgers.

Hamburgers of 130 grams each are subsequently shaped with the use of a mould.

The hamburgers are blast frozen for 90 minutes and then transferred to a normal freezer. The hamburgers are stored in the freezer for at least 3 nights before use. The day before the cooking the hamburgers are removed from the freezer and placed in the fridge to thaw. The starting temperature of the burgers before cooking is 7° C. A cooking plate is set at 160° C., and burgers are cooked for 3 minutes at each side alternately until core temperature was above 75° C.

TABLE 3
Ingredients of vega-burger using glucoamylase in the binder phase.

| | | | GPA + | GPA + | GPA + |
|---|---|---|---|---|---|
| Ingredients (in weight %) | MC | GPA | AM | HU | EWP |
| TVP TU-crumble caramel | 14 | 14 | 14 | 14 | 14 |
| 180 (ADM) | | | | | |
| Caramelized sugar ncs 23p | 0.2 | 0.2 | 0.2 | 0.2 | 0.2 |
| from Buisman | | | | | |
| Beet root from Diana | 0.3 | 0.3 | 0.3 | 0.3 | 0.3 |
| water 1 (hydration) | 32.5 | 32.5 | 32.5 | 32.5 | 32.5 |
| Methylcellulose (methocel), | 1.5 | | | | |
| texturant 1 | | | | | |
| Gellan Gum HA (Gellaneer | 0.6 | 0.6 | 0.6 | 0.6 | 0.6 |
| HD ™; DSM), texturant 1 | | | | | |
| Pectin APC200Y (Andre | | 0.5 | 0.5 | 0.5 | 0.5 |
| Pectin), texturant 1 | | | | | |
| Aglufiber FAS (Silvateam), | | 1.0 | 1.0 | 1.0 | 1.0 |
| texturant 2 | | | | | |
| Amigase Mega L | | | 10 | | |
| Hunan HY01 | | | | 10 | |
| Egg white powder | | | | | 3 |
| sunflower oil from local | 8 | 8 | 8 | 8 | 8 |
| brand | | | | | |
| Ice cold water 2 | 35 | 35 | 29.4 | 29.4 | 35 |
| Soy protein isolate | 3 | 3 | | | |
| (myprotein.com) | | | | | |
| Salt | 0.67 | 0.67 | 0.67 | 0.67 | 0.67 |
| Flavors | 1.6 | 1.6 | 1.6 | 1.6 | 1.6 |
| Coconut fat | 2 | 2 | 2 | 2 | 2 |

MC: methylcellulose; GPA: gellan, pectin, Aglufiber; AM: Amigase Mega L; HU: Hunan HY01; EWP: Egg white protein.

Hardness of samples of the raw and cooked burgers was determined by TPA as described above with a probe of 25 mm. Raw burgers were analyzed at 10° C., cooked burgers at 50° C. Relative increase of hardness upon cooking is an indicator of the effectiveness of the tested ingredients in heat gelation. It was calculated by dividing the hardness (g) of the cooked burgers by the hardness (g) of the raw burgers. Results are indicated in Table 4 and show that different samples containing glucoamylase protein led to a clear increase in hardness upon cooking, even higher than the relative increase in hardness with methylcellulose or egg-white. A good increase in firmness of the cooked samples was also detected by a trained panel.

TABLE 4
Fold increase in hardness of vegan burgers upon cooking.

| | |
|---|---|
| Methylcellulose/gellan | 3.1 |
| gellan/pectin/aglufiber (GPA) | 1.2 |
| GPA + Amigase Mega L | 5.5 |
| GPA + Hunan HY01 | 6.6 |
| GPA + egg white powder | 3.4 |

### FIGURE LEGENDS

FIG. 1: Rheological assessment of a 2.5% and 10% glucoamylase (Amigase Mega L) protein solution in water in comparison to egg white at the same dilution. A; Complex modulus (G* in Pa) versus temperature. B; Complex modulus (G* in Pa) versus strain (%) in heat-set gel performed at 25° C.

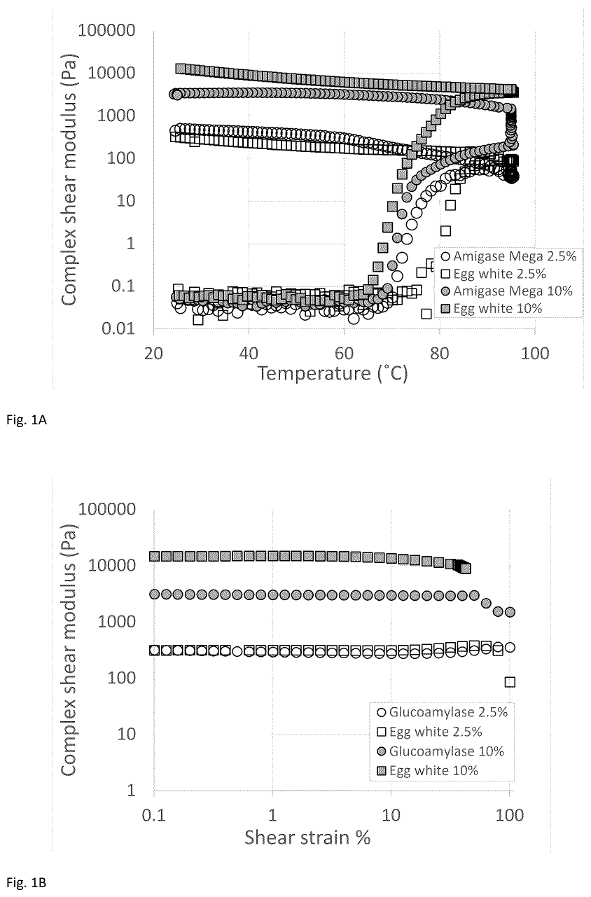

FIG. 2: Rheological assessment of a 10% glucoamylase protein (Amigase Mega L and Sunbake AMG NG), and egg white at the same protein concentration in water. A; Complex modulus (G* in Pa) versus temperature. B; Complex modulus (G* in Pa) versus strain (%) in heat-set gel performed at 25° C.

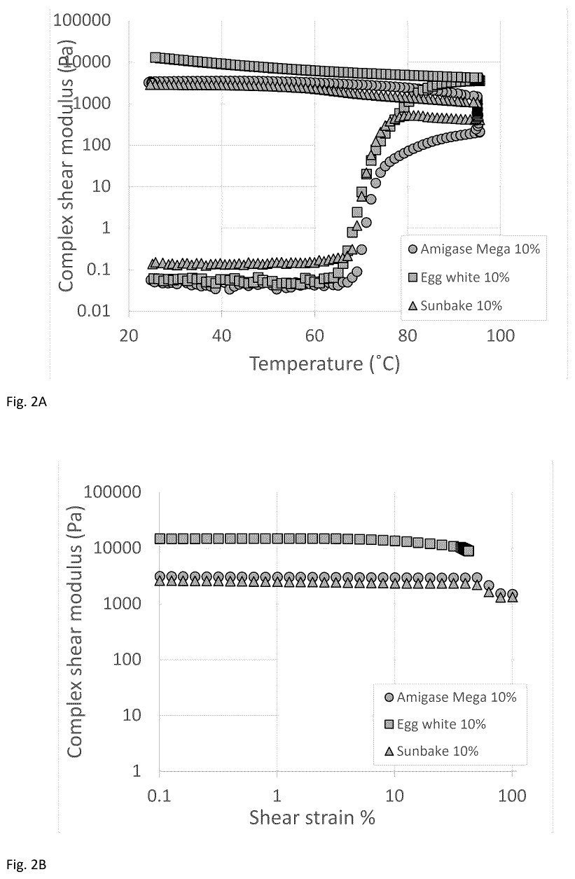

1. A method for preparing a non-animal protein food product or a cultured meat food product comprising
adding a thermolabile glucoamylase protein preparation to an ingredient used in the preparation of said food product and processing the obtained glucoamylase protein preparation and ingredient composition into a non-animal protein food product or a cultured meat food product.

2. The method according to claim 1, wherein said glucoamylase protein preparation provides binding properties to the food product when said glucoamylase protein preparation and ingredient composition or said food product is heated to at least 65 degrees Celsius.

3. The method according to claim 1, further comprising heating the food product to at least 65 degrees Celsius.

4. The method according to claim 1, wherein said glucoamylase protein preparation comprises
a native glucoamylase protein;
a fungal glucoamylase protein;
an Aspergillus glucoamylase protein;
an Aspergillus niger glucoamylase protein;
a classical (non-GMO) glucoamylase protein;
an extracellularly produced glucoamylase protein; and/or
any combination thereof.

5. The method according to claim 1, wherein said non-animal protein is plant protein, microbial protein, or algal protein.

6. The method according to claim 5, wherein said microbial protein is fungal protein or bacterial protein.

7. The method according to claim 1, wherein said non-animal protein food product or said cultured meat food product is a meat-alternative food product or a fish alternative food product.

8. The method according to claim 1, wherein the amount of glucoamylase protein is 0.025 to 10%, optionally 0.05 to 10%, or optionally 0.25 to 10% (relative to the mass of the food product).

9. A food product obtainable by the method of claim 1.

10. A meat or fish alternative food product comprising non-animal protein or cultured meat and a thermolabile glucoamylase protein preparation.

11. The meat or fish alternative food product according to claim 10, further comprising non-animal fat.

12. A meat or fish alternative which comprises texturized vegetable protein, non-animal protein, water, flavour and a binder system, wherein the binder system comprises a thermolabile glucoamylase protein preparation.

13. A method of at least partly replacing a binder in a food product, the method comprising adding a thermolabile glucoamylase protein preparation to said food product.

14. The method of claim 13, wherein all binder is replaced by said glucoamylase protein preparation.

15. The method of claim 13, wherein said binder is methylcellulose.

16. The method of claim 13, wherein said food product is a meat-alternative food product or a fish-alternative food product.

17. The method of claim 1, wherein said thermolabile glucoamylase protein preparation provides gelling properties to the food product when said glucoamylase protein preparation and ingredient composition or said food product is heated to at least 65 degrees Celsius.
